(19) United States
(12) Patent Application Publication
(10) Pub. No.: US 2014/0271904 A1
(43) Pub. Date: Sep. 18, 2014

(54) METHODS FOR PREPARING TISSUE IMPLANTS

(71) Applicant: CryoLife, Inc., Kennesaw, GA (US)

(72) Inventors: Joseph Hamby, Woodstock, GA (US); Steven Goldstein, Atlanta, GA (US); Valerie Rohde, Dallas, GA (US)

(73) Assignee: CryoLife, Inc., Kennesaw, GA (US)

(21) Appl. No.: 14/200,422

(22) Filed: Mar. 7, 2014

Related U.S. Application Data
(60) Provisional application No. 61/792,842, filed on Mar. 15, 2013.

(51) Int. Cl. A61K 35/12

(52) U.S. Cl. CPC A61K 35/12
USPC 424/520

(57) ABSTRACT

A method is provided for preparing a biological tissue for implantation. The method includes providing a biological tissue from a human or animal donor, treating the biological tissue with an antiviral treatment formulation, lysing the biological tissue, decellularizing the biological tissue with a decellularization treatment formulation, and decontaminating the biological tissue with an alkaline alcohol solution. The antiviral treatment formulation may include a solution of peracetic acid and an alcohol, wherein the peracetic acid is present in the antiviral treatment formulation at a concentration from about 0.03% to about 1.2% (v/v). The decellularization solution may include a solution of a polar aprotic solvent, such as dimethyl sulfoxide, benzyl alcohol and ethanol.

### CROSS-REFERENCE TO RELATED APPLICATIONS

The present application claims priority to and benefit of U.S. Provisional Application No. 61/792,842, filed Mar. 15, 2013, which is incorporated by reference herein in its entirety.

### FIELD

This application is generally in the field of methods for preparing biological tissues for implantation. In particular, it relates to methods for treating biological tissues for implantation without use of antibiotics.

### BACKGROUND

Human allograft and other animal-derived, tissue-based implantable materials generally undergo a series of processing steps, which may include procurement, transportation, decontamination, freezing, storage, thawing, terminal sterilization, and transplantation steps. Various decontamination methods are known in the art, including chemical fixation, chemical decontamination, ionizing radiation, and treatment with antibiotic compositions. These processes generally are complex and time-consuming and require use of antibiotics and numerous antiviral treatments to obtain the necessary 6- to 10-log viral reduction required for medical implants. For example, existing antiviral treatments generally require multiple antiviral treatments, which may include solution-based antiviral treatments. For solution-based treatments, the solutions often contain materials resulting in hazardous waste streams or result in damage to the tissue material. Existing methods often are also ineffective at removing all lipid bound immunologically active moieties, resulting in undesirable immunologic responses upon implantation of the tissue implant. For example, existing processes may leave residual alpha-Gal epitopes in areas associated with lipid, and more specifically waxy, secretions.

As such, there remains a need for better processes for treating biological tissues for implantation that, for example, provide the requisite decontamination and decellularization of the biological tissue without impairing the tissue viability. In particular, there is a need to develop improved processes for viral reduction that are effective, use fewer steps than existing methods, and that do not negatively affect the tissue being processed. It also is desirable that such methods reduce or eliminate the production of hazardous waste streams.

### SUMMARY

Embodiments provided herein address the above-described needs by providing methods for preparing a biological tissue for implantation.

In an embodiment, a method for preparing a biological tissue for implantation is provided that includes (i) providing a biological tissue; contacting the biological tissue with an antiviral treatment formulation comprising an aqueous solution of peracetic acid and an alcohol; (ii) decellularizing the biological tissue; and (iii) decontaminating the biological tissue with an alkaline alcohol solution. Desirably, the peracetic acid is present in the antiviral treatment formulation at a concentration from about 0.03% to about 1.2% (v/v).

In another embodiment, a method is provided for preparing a biological tissue for implantation without the use of antibiotics that includes (i) providing a biological tissue; (ii) contacting the biological tissue with an antiviral treatment formulation comprising a solution of peracetic acid and an alcohol, wherein the peracetic acid is present in the antiviral treatment formulation at a concentration from about 0.03% to about 1.2% (v/v); (iii) lysing the biological tissue; (iv) decellularizing the biological tissue with a decellularization solution comprising ethanol, dimethyl sulfoxide, and benzyl alcohol; and (v) decontaminating the biological tissue with an alkaline alcohol solution.

These and other aspects and embodiments of the disclosed methods are described more fully in the detailed description.

### BRIEF DESCRIPTION OF THE DRAWINGS

FIG. 1 is a flowchart illustrating a method for preparation of implantable tissue materials according to an embodiment.

### DETAILED DESCRIPTION

Improved processes for the preparation of implantable tissue materials have been developed. The terms “biological tissue” and “tissue material” are used interchangeably to refer to mammalian tissues, such as non-human mammalian tissues, which are suitable for use to prepare allograft or xenograft tissue for implantation in humans in need of such grafts. In embodiments, the biological tissue is derived from a mammalian tissue comprising collagen.

The method generally includes pre-treating a tissue material with an antiviral treatment formulation, treating the tissue with a hypotonic solution to lyse the cells in the tissue material, decellularizing the tissue material with a decellularization treatment formulation comprising a polar aprotic solvent and an antimicrobial, and treating the tissue with an alkaline alcohol. The method for preparing a tissue material for implantation may further include treating the tissue material with a bleaching solution and a residual reduction treatment before vacuum sealing the tissue material in a package. The tissue material may be thereafter frozen and/or sterilized by ionizing radiation. Alternatively, in some embodiments, the tissue material may be chemically sterilized prior to packaging or after packaging, e.g., prior to vacuum sealing. The tissue implant may be stored cryopreserved, frozen, refrigerated, or at ambient temperature storage conditions depending on the final configuration of the tissue implant.

In embodiments, the antiviral treatment formulation comprises an acidic solution of peracetic acid and alcohol and is effective to provide the needed viral reduction while substantially maintaining the physiological characteristics of the biological tissue. The enhanced effectiveness of the antiviral treatment process may be the result of employing a combination of the oxidative power of peracetic acid and the solvent properties of the alcohol at the low pH, advantageously providing functionality against both enveloped and non-enveloped viruses.

In embodiments, the polar aprotic solvent of the decellularization treatment formulation comprises, for example, dimethyl sulfoxide (DMSO), in an amount effective to reduce antigenicity of the biological tissue that may result from lipid bound immunologically active moieties. The enhanced effectiveness is believed to be imparted by dissolving the glycolipids associated with the immunologically reactive molecules, allowing for their removal during subsequent processing.

As used herein, the term “amounts effective,” “effective amount,” or the like as used in reference to one or more of the antiviral agents means that the agent(s) is/are present at a sufficient concentration such that the composition substantially reduces the viral load but does not substantially negatively alter physiological characteristics of the tissue which would affect the tissue's suitability for use as a tissue implant. Suitability may be determined by evaluating physiological characteristics of the tissue including, but not limited to, viability, biomechanics, denaturation temperature, and microscopic evaluation of the tissue. Thus, “effective amounts” can be determined by dose response testing as is known in the art using standard microbiological tests and viability tests such as those known in the art or described below. Similarly, the term “amounts effective,” “effective amount,” or the like as used in reference to one or more of the deceullarization agents means that the agent(s) is/are present at a sufficient concentration such that the composition substantially dissolves the glycolipids associated with immunological reactive molecules but does not substantially negatively alter physiological characteristics of the tissue which would affect the tissue's suitability for use as a tissue implant.

As used herein, the term “substantially inactivating viruses” means that the composition completely inactivates viruses in at least 90%, preferably at least 99%, most preferably at least 99.9%, of the tissues treated with the composition. Desirably, substantially inactivating viruses results in a 6- to 10-log viral reduction of the viral load of the biological tissue.

As used herein, the term “substantially dissolves glycolipids” means that the composition completely dissolves glycolipids associated with immunological reactive molecules in at least 90%, preferably at least 99%, most preferably at least 99.9%, of the tissues treated with the composition. “Completely dissolves glycolipids” means that residual alpha-gal epitopes are not detectable by standard immunohistochemical assays after the tissue has been treated with the composition.

As used herein the term “substantially maintaining the physiological characteristics of the tissue” means that the treatment composition does not adversely affect the physiological characteristics of the tissue that render the tissue suitable for use in reconstruction, repair or replacement in human patients or other mammalian subjects. As such, the physiological characteristics that are maintained depend on the physiological characteristics of the tissue subject to the decontamination treatment with the composition. For example, for decellularized tissue grafts, the term “substantially maintaining the physiological characteristics of the tissue” primarily encompasses maintaining the biomechanical properties of the tissue without denaturing collagen, elastin, and other protein components of the tissue structure.

In some embodiments, as explained above, the antiviral treatment formulation is effective at substantially inactivating viruses on the tissue while substantially maintaining the viability of the tissue. Similarly, in some embodiments, the decellularization treatment formulation is effective at substantially dissolving the glycolipids associated with immunological reactive molecules while substantially maintaining the viability of the tissue. Viability can be measured in a number of ways. In one embodiment, the tissue is incubated with a radioactively-labeled amino acid, and the incorporation of the amino acid into proteins is monitored by counting disintegrations per minute (DPM) per unit of tissue. Accordingly, as used herein, the term “substantially maintaining the viability” means that tissue that has been treated with the antiviral formulation incorporates at least about 85% of the DPM per unit tissue, as compared to tissue that is not treated with the formulation.

In embodiments, a “tissue material for implantation” or “biological tissue for implantation”, are used interchangeably to refer to a native, normally cellular tissue that has been procured from a mammal, and mechanically cleaned of attendant tissues and chemically cleaned of cells, cellular debris and rendered substantially free of non-collagenous extracellular matrix components. The tissue material for implantation, while substantially free and purified of non-collagenous components, maintains much of its native matrix structure, organization, strength, and shape.

### I. Methods

Methods are provided for preparing tissue implants for implantation, particularly decellularized tissue implants having reduced antigenicity and cytotoxicity. An exemplary embodiment includes obtaining a starting tissue material, treating the tissue material with an antiviral treatment formulation comprising peracetic acid, treating the tissue material with a hypotonic solution, treating the tissue material with a decellularization solution comprising a polar aprotic solvent, treating the tissue material with an alkaline alcohol, treating the tissue material with chlorine dioxide, treating the tissue material with an antioxidant, and vacuum sealing the tissue material in a package. The implantable tissue material may be thereafter frozen and/or sterilized by ionizing radiation. Alternatively, in some embodiments, the implantable tissue material may be chemically sterilized prior to packaging or after packaging, e.g., prior to vacuum sealing. The implantable tissue material may be stored cryopreserved, frozen, refrigerated, or at ambient temperature storage conditions depending on the final configuration of the implantable tissue material.

For example, as illustrated in FIG. 1, an embodiment of a method 10 for preparing a tissue implant for implantation may include a series of treatment steps that transform a harvested, starting tissue material into a tissue implant, which may be implanted in a patient for various purposes, particular therapeutic, prophylactic, reconstructive, or other medical purposes. Initially, a starting tissue material may be obtained in accordance with step 12. The starting tissue material may be harvested from a variety of animal donor tissues, including human or other mammalian sources. For example, the starting tissue material may comprise a portion of dermis harvested from a porcine donor.

The starting tissue material is treated with an antiviral treatment formulation in accordance with step 14. Desirably, the antiviral treatment formulation may be an acidic aqueous solution including peracetic acid and an alcohol. The tissue material may then be lysed in a hypotonic solution in accordance with step 16. The step of lysing the tissue material may cause the cells of the tissue material to osmotically rupture or otherwise render the cells susceptible to decellularization by subsequent processing steps.

The tissue material then may be treated with a decellularization treatment formulation that includes a polar aprotic solvent and an antimicrobial agent, e.g., benzyl alcohol, to decellularize and decontaminate the tissue material in accordance with step 18. The tissue material then may be decontaminated further by treatment, for example, with an alkaline alcohol solution in accordance with step 20. The alkaline alcohol treatment may change the texture of the tissue material, providing tactile qualities that make it easier to handle during surgery. Thereafter, the tissue material may be rinsed for residual reduction in accordance with step 22, treated with a bleaching solution in accordance with step 24, followed by one or more washings with a buffer solution for further residual reduction in accordance with step 26. The tissue material then may be vacuum sealed in a package in accordance with step 28. In certain embodiments, the tissue material is vacuum sealed in an antioxidant treatment solution. The tissue material may then be irradiated with gamma or electron beam radiation in accordance with step 30. The resulting implantable tissue materials desirably have a reduced cytotoxicity and antigenicity as compared to implantable tissue materials prepared using prior art methods.

In some embodiments, the methods described herein are performed by moving the tissue material between different containers that contain the various treatment solutions. In other embodiments, the methods described herein are performed by maintaining the tissue material in a container, and serially filling the container with a series of treatment solutions. For example, the various treatment solutions may be circulated through the container sequentially. In some embodiments, the container is filled with a treatment solution, then the container is drained, and a second treatment solution is added. In some embodiments, the methods are performed by a combination of the foregoing. Moreover, one or more steps of the process may be partially or totally automated. To facilitate such processes, the tissue may be secured to a tissue holding device to ensure uniform exposure of the tissue to the treatment solution. For example, the tissue may be attached to a frame. The frame may maintain the tissue in a plane so that the tissue does not fold during treatment.

It is contemplated that methods of preparing tissue implants for implantation may comprise various combinations of the foregoing steps consistent with the teachings of the present disclosure. Furthermore, although the present methods are described in the context of unfixed allograft and xenograft tissue materials, aspects of the disclosed methods may also be utilized with biosynthetic material and/or fixed biologics. That is, the source tissues may be produced using in vitro tissue synthesis techniques as well as recombinant means known in the art. For example, the described antiviral treatment and alkaline alcohol treatment, may be used in treatment processes for biosynthetic and fixed tissues. Other variations would be obvious to one of ordinary skill in the art in view of the present disclosure. Various steps for preparing a tissue material for implantation are described in greater detail below.

A. Tissue Procurement

Tissue materials suitable for use in the present methods include those appropriate for implantation into humans or animals. Tissue material for transplantation may be harvested from various sources. Tissues can be human or non-human, e.g., bovine, porcine, equine, ovine, macropodidae (e.g., kangaroo) or non-human primate in origin. Such starting tissue materials include tissue materials that are derived from human or-nonhuman sources. The present methods may be used to prepare allograft or xenograft tissue for implantation in humans.

While the present invention is often exemplified by reference to epithelial tissues, particularly porcine dermis, the present methods are applicable to other tissues as well, particularly soft tissues. Specifically, the present methods may be employed to prepare connective tissue, muscle tissue, nervous tissue or vascular tissue for implantation. Examples of suitable epithelial, mesothelial, and endothelial tissues include skin tissues and the tissues that line body cavities and lumen, including, but not limited to, pericardium, peritoneum, pleura, blood vessels (e.g., veins and arteries), stomach, intestines, esophagus, Fallopian tubes, endometrium, cervix, vagina, trachea, bronchioles, tympanic membrane, ureter, umbilical cord and bladder. Other tissues that may be used include tendons, ligaments, fascia, dura mater, diaphragm, and heart valves.

In an exemplary embodiment, the tissue material comprises porcine dermis. The porcine dermis may be sourced and harvested in accordance with applicable regulatory requirements. The tissue may be thereafter transported in a suitable physiological buffer solution. For example, the tissue may be transported in phosphate-buffered saline (PBS) in refrigerated conditions.

In embodiments, before subjecting the tissue material to the antiviral, decellularization, and decontamination treatments described herein, the tissue material may be trimmed to a desired size and shape. Before trimming the tissue material, a portion of the tissue material may be first selected that has desired thickness and physical properties and is generally free from defects. The selected portion of the tissue material may be cut to the desired shape and size. For example, a patch may be cut from the tissue material in the shape of a circle, ellipse, oval, or rectangle. In a preferred embodiment, the tissue material may be cut in a substantially circular shape, for example, a circle having a diameter of about 4 cm to about 12 cm, or more preferably about 5 cm to about 10 cm, or about 7 cm. In another embodiment, the tissue material may be cut to form a patch that is substantially elliptical in shape, such as a 5 cm by 8 cm ellipse. Other shapes may be used including regular and irregular polygons, curvilinear shapes, and combinations thereof.

In some embodiments, a portion of tissue material may be selected that is free of non-target or non-desired tissue elements and is substantially uniform in thickness. For example, for porcine dermis it is generally possible to cut a 10 cm by a 20 cm sheet from the dermis that is free of extraneous connective tissue and substantially uniform in thickness. In one embodiment, an entire sheet of tissue material subjected to the antiviral, decellularization, and decontamination treatments described herein. In other embodiments, the sheet is cut into smaller portions, e.g., patches, before being subjected to the antiviral, decellularization, and decontamination treatments described herein.

In some embodiments, the tissue material is cut with a laser. In certain embodiments, the laser is computer-controlled to cut the desired shape based on a computer program. The controller may automatically adjust the direction, power, and speed of the laser and/or automatically adjust the orientation or location of the tissue material relative to the laser. Laser cutting may result in a Heat Affected Zone (HAZ) at the edge of the cut. In some embodiments, the HAZ may be removed by washing the tissue material in a solution, such as sodium ascorbate. In some embodiments, HAZ is avoided or minimized by using an ultra-fast laser, i.e., a laser with a short pulse duration, such as a femtosecond laser. In other embodiments, the tissue material may be cut using a high-velocity water stream, an arc cutter, a clicker press, or manually with a bladed instrument.

Other biological tissue materials may be trimmed to form patches and/or other tissue articles of various shapes and sizes. The specific shape and size will depend on the intended use of the patch or article.

B. Antiviral Treatment

In embodiments, the tissue material undergoes treatment with an antiviral treatment formulation prior to decellularization of the tissue material. Antiviral treatment formulations for preparing the tissue material for implantation desirably comprise an aqueous solution of peracetic acid and an alcohol and are characterized by an acidic pH from about 4.7 to about 5.0.

The peracetic acid may be present in the antiviral treatment formulation at a concentration from about 0.03% to about 1.2% (v/v) and the alcohol may be present in the antiviral treatment formulation at a concentration from about 10% to about 20% (v/v). In exemplary embodiments, the antiviral treatment formulation comprises peracetic acid at a concentration from about 0.03% to about 0.5%, from about 0.03% to about 0.1%, from about 0.03% to about 0.075%, or about 0.05% (v/v).

The alcohol of the antiviral treatment formulation desirably comprises ethanol, although other suitable alcohols also may be used. Non-limiting examples of other alcohols suitable for use in the antiviral treatment formulation include methanol, propanol, isopropanol, and combinations thereof.

The antiviral treatment formulation may further comprise other suitable components, such as salts. For example, in an embodiment the antiviral treatment formulation comprises an aqueous sodium chloride solution with a concentration from about 0.9% to about 1M as a solvent.

In embodiments, the step of treating the tissue material with the antiviral treatment formulation comprises contacting the tissue material with the antiviral treatment formulation for a period from about 30 minutes to about 240 minutes (4 hours), from about 30 minutes to about 120 minutes (2 hours), or from about 60 minutes to about 90 minutes. The antiviral treatment may be conducted at room temperature or at a temperature near but below the flammability level of the alcohol in the aqueous solution.

C. Hypotonic Lysis

After the antiviral treatment, the tissue material may be placed in a hypotonic solution in order to affect cell lysis. When placed in a hypotonic solution, water may be drawn into the cells of the tissue material through the cell membrane via osmosis. This may cause the cells of the tissue material to swell and burst. In some embodiments, the tissue material may be retained in the hypotonic solution for a period of time sufficient to cause the cells of the starting tissue material to osmotically rupture or otherwise render the cells susceptible to decellularization by subsequent processing steps.

The term “decellularization” as used herein refers to the destruction of the cells of a tissue material. Various observational and/or quantitative methods are known for evaluating decellularization and measuring the degree thereof. For example, the degree of decellularization may be measured by performing a hematoxylin stain of nuclei present in a starting tissue material and the processed tissue material and comparing the results. A tissue material may be considered “essentially acellular” if the tissue material comprises at least 70% fewer hematoxylin stainings than the starting cellular tissue material. More preferably, a decellularized tissue material comprises 95% fewer, or even more preferably 99% fewer hematoxylin stainings than the starting cellular tissue material.

Solutions for effecting cell lysis may include water or a solution having a solute (e.g., a salt such as NaCl) concentration of up to about 80 millimolar (for example, a 10-20 or 20-40 mM NaCl solution). Lysis can be performed, for example, at a temperature in the range of 20° C. to 40° C., preferably about 37° C. The tissue material may be maintained in the hypotonic solution for about 4 hours to about 10 days, or preferably about 4 hours to about 8 hours. The amount of time needed to achieve cell lysis may depend on the temperature at which lysis is performed. For example, at higher temperatures, lysis may be achieved in a shorter period of time than when treated at a lower temperature.

D. Decellularization

Following lysis, the tissue material may be decellularized by treatment with a decellularization treatment formulation comprising a polar aprotic solvent. Suitable polar aprotic solvents for use in embodiments of the present application are characterized as antigenicity-reducing agents, and are effective at dissolving glycolipids associated with immunological reactive molecules. Non-limiting examples of suitable polar aprotic solvents include acetone, dimethyl sulfoxide, dimethylformamide, dioxane, and hexamethylphosphoratriamide. In some embodiments, the decellularization treatment formulation comprises the polar aprotic solvent in a concentration of about 5% to about 25%, from about 5% to about 20%, or more preferably about 10% to about 20% (v/v).

The decellularization treatment formulation may further include one or more antimicrobial agents effective for preventing a bacterial bloom during decellularization. For example, the decellularization treatment formulation may include one or more non-antibiotic antimicrobial agents such as preservatives and/or antiseptic agents. Exemplary preservatives include, but are not limited to, benzyl alcohol, sodium metabisulfite and/or methylparaben. Chlorhexidine is an exemplary antiseptic agent. In some embodiments, the decellularization treatment formulation comprises about 0.8% to about 2.0% (v/v) benzyl alcohol. Benzyl alcohol is a bacteriostatic agent and has been discovered to be effective for preventing the growth of bacteria, particularly gram negative bacteria. The decellularization treatment formulation, without the benzyl alcohol, may provide conditions that allow for the growth of viable bacteria remaining from previous treatment steps. Benzyl alcohol provides a bacteriostatic/bacteriocidal agent to the decellularization treatment formulation that does not negatively affect the process outcome.

The decellularization treatment formulation may further include one or more other components. For example, in embodiments the decellularization treatment formulation may comprise ethanol in a concentration from about 5% to about 10% (v/v).

The tissue material may be incubated in the decellularization treatment formulation at a temperature from about 30° C. to about 40° C., from about 30° C. to about 39° C., preferably about 37° C. The tissue material may be incubated in the decellularization treatment formulation for about 24 hours to about 48 hours or for any period of time sufficient to degrade the cell nuclei material.

The decellularized tissue material desirably is rendered essentially acellular while substantially maintaining the physiological characteristics of the tissue. Thus, a decellularized tissue material primarily encompasses a tissue that is rendered essentially acellular without denaturing the collagen, elastin, and other protein components of the tissue structure.

E. Alkaline Alcohol Treatment

After decellularization, the tissue material may undergo further decontamination treatments. In one embodiment, the tissue material is contacted with an alkaline alcohol solution. The alkaline alcohol solution may be effective for reducing the nuclease content of the tissue material, reducing the viral load of the tissue material and/or altering the textural quality of the tissue material.

The alcohol component of the alkaline alcohol solution may include isopropanol, methanol or ethanol, while the alkaline component of the alkaline alcohol solution may include a hydroxide salt. For example, the alkaline alcohol solution may include ethanol in a concentration of about 72% to about 88% (v/v). In some embodiments, the alkaline solution is about 0.016M to about 0.022M sodium hydroxide. In an exemplary embodiment, the alkaline alcohol solution includes about 80% (v/v) ethanol and about 0.02M sodium hydroxide in about 0.9% saline. The tissue material may be treated in the alkaline alcohol solution for about 60 minutes to about 3 hours at a temperature from about 30° C. to about 40° C., preferably from about 35° C. to about 39° C.

F. Residual Reduction and Bleaching

The decontaminated and decellularized tissue material may undergo one or more washes for further residual reduction. In embodiments, the residual reduction includes a series of washes of the decontaminated and decellularized tissue material with water and/or a buffer solution (e.g., Tris buffer). For example, in an embodiment the residual reduction includes rinsing the tissue material with deionized water at room temperature in two steps.

The tissue material also may undergo a bleaching treatment in series with the residual reduction for aesthetic reasons. For example, the tissue material may be contacted with an aqueous solution including about 0.05% to about 0.1% (v/v) peracetic acid at room temperature for about 15 minutes to about 30 minutes. In an exemplary embodiment, the tissue material is contacted with a 0.05% (v/v) solution of peracetic acid at room temperature for 30 minutes.

After bleaching, the tissue material may undergo further treatment for residual reduction. For example, the tissue material may be contacted with water and/or a buffer solution at room temperature for about 60 minutes to about 90 minutes.

G. Vacuum Sealing

The tissue material may then be vacuum sealed in a container to reduce further exposure of the tissue material to oxygen. In some embodiments, the tissue material is vacuum sealed in a package with an antioxidant solution, e.g., an ascorbic acid or one of its sodium, potassium, or calcium salts. The antioxidants in the solution may have a radioprotective function that prevents oxidation and mitigates free radical damage from gamma irradiation.

Preferably, the tissue material is vacuum sealed in package that is dimensioned to allow the tissue material to be maintained in a flat and unfolded state when sealed. Various types of vacuum systems may be employed to create the seal. For example, a venturi or a vacuum pump may be used to remove air from the package prior to initiating a heat seal. In some embodiments, a vacuum level of about 13 in Hg is achieved before the heat seal is created. In some embodiments, an evacuate-flush-evacuate cycle is used to remove oxygen from the package. For example, the package may be subjected to a vacuum to evacuate the air from the package, then filled with an inert gas such as nitrogen or argon, and then subjected to a vacuum once again to evacuate the inert gas (e.g., nitrogen or argon) from the package.

The removal of air from the package prevents air pockets from developing between the tissue implant and the pouch and reduces free radical formation when then package is subjected to gamma irradiation. The vacuum also advantageously causes the package to maintain the tissue material in a flat and stationary position during transport and prevents the formation of permanent folds/creases when the tissue is subjected to irradiation.

H. Packaging of Tissue for Transport

The tissue material may thereafter be packaged for transport. In some embodiments, a double pouch configuration is employed. The vacuum sealed inner pouch may comprise a clear film material. For example, the inner pouch may be formed of a multilayer laminate film with an inner heat-sealable layer and one or more outer layers having good oxygen barrier properties. The inner pouch may have a low vapor transmission level. In some embodiments, the heat sealed inner pouch is stored in an outer pouch, which may comprise foil or a metalized polymer film. The outer pouch preferably does not allow for any measureable vapor transmission. The double pouch configuration may have a shelf life of at least two years under ambient conditions. Advantageously, the tissue material may be stored without refrigeration or freezing. The packaging, e.g., the inner pouch and outer pouch, is preferably radiation stable.

In some embodiments, the tissue material is transported cold but at a temperature greater than the freezing point of the antioxidant treatment solution so that the tissue material is not allowed to freeze during transport.

I. Sterilization/Crosslinking

The tissue may be terminally sterilized while packaged. In some embodiments, the tissue implant is subjected to gamma or electron beam irradiation in a radiation dosage sufficient to further reduce the microbial content of the tissue material. For example, the tissue may be subjected to radiation of about 15 to about 40 kGy. In some embodiments, the tissue implant is sterilized with a radiation dosage sufficient to achieve a Sterility Assurance Level (SAL) of 10⁻⁶.

In other embodiments, the tissue may be terminally sterilized with chemical sterilants or may be treated with crosslinking agents or other chemicals prior to or after packaging. In some embodiments, the tissue implant is contacted with the chemicals in sufficient concentration and/or amount to further reduce the microbial content of the tissue material. For example, the tissue may be treated with ethylene oxide, formaldehyde, hydrogen peroxide, ozone, glutaraldehyde, propiolactone, o-phthaldehyde, propylene oxide, mercurials, phenols, chlorine, hypochlorite, iodophore, peracetic acid, superoxidized water, chlorhexidine, detergents, supercritical fluids (e.g., supercritical CO₂), quaternary ammonium compounds, silver, kathon, inactine (PEN110), ultrasonication, pressure cycling, and/or steam.

J. Unpackage and Use

Immediately prior to use, the tissue may be removed from the packaging, for example, by peeling away, tearing, or cutting the outer pouch and inner pouch of the packaging. In embodiments in which the tissue is stored in the antioxidant treatment solution, the tissue is ready for use when removed from the inner pouch without requiring rinsing or re-hydration.

The tissue may then be implanted at the desired location, in a patient. For example, the tissue implant may be used to reinforce soft tissues where weakness exists. In some embodiments, the tissue may be used to reinforce soft tissues repaired by sutures or by suture anchors, e.g., as part of a tendon repair surgery. In some embodiments, the tissue is implanted as a patch to repair defects of the abdominal or thoracic wall. The tissue implant may also be implanted in patch form to repair the heart, a rectal or a vaginal prolapse, or it may be implanted for reconstruction of the pelvic floor. The tissue may be implanted to patch hernias, as reinforcement for a suture-line, or it may be used in breast or other reconstructive and/or cosmetic procedures. The tissue implant may be utilized as a vascular patch as in the procedure of endaterectomy. The tissue implant may also be used in urinary systems. In some embodiments, the tissue is implanted as to reinforce a rotator cuff, a patella, an Achilles, a bicep, a quadriceps, or other tendon or ligament.

### II. Tissue Implants

Tissue implants produced by the foregoing methods may possess many desirable properties for reconstructive and reinforcing applications, such as suitable mechanical strength, biocompatibility, and the ability to support recellularization in vivo. The tissue implants may further provide good physical support for repair, and a biological scaffold for healing.

In some embodiments, the tissue implants are essentially acellular. In certain embodiments, the tissue implants have at least 95% fewer intact cells, or 99% fewer intact cells than the naturally occurring biological material. In some embodiments, the tissue implants consist essentially of structural proteins, e.g., collagen and elastin. In some embodiments, the tissue implants are unfixed, i.e., not chemically cross-linked. The tissue may also be cross-linked, such as with 1-ethyl-3-(3-dimethylaminopropyl) carbodiimide (“EDC”). The tissue implants may therefore be remodelable. The tissue implants may also support healing by providing a tissue matrix that is capable of recellularizing and remodeling.

In some embodiments, the tissue implant may comprise decellularized tissue, such as decellularized human, bovine, equine, macropodidae or porcine pericardium, dermis, omentum, amniotic membrane, peritoneum, or urinary bladder. The tissue implants may also comprise decellularized tissue materials of other origins, including vascular tissues, orthopedic tissues, connective tissues, cardiac tissues, and other tissues that are sheet-like in configuration.

The tissue implants may be of uniform thickness and physical properties. For example, the tissue implant may be a patch, such as a patch in the shape of a circle, ellipse, oval, or rectangle. In a preferred embodiment, the tissue implant may be a patch that is substantially circular shape, for example, a circle having a diameter of about 4 cm to about 12 cm, or more preferably about 5 cm to about 10 cm, or about 7 cm. In another embodiment, the tissue implant may be in the form of a patch that is substantially elliptical in shape, such as a 5 cm by 8 cm ellipse. The tissue implant may be formed into a patch of various other shapes including regular and irregular polygons, curvilinear shapes, and combinations thereof.

Tissue implants produced by the foregoing methods have been found to be non-cytotoxic, non-sensitizing, non-irritating, non-genotoxic, non-pyrogenic, non-hemolytic, and do not promote system toxicity using antibiotic-free processes without requiring use of treatments that produce undesirable hazardous waste. Biomechanical evaluations of the tissue implants have demonstrated high tensile, burst, and tear resistance, as well as suitable suture retention strengths.

The denaturation temperatures of tissue implants produced by the foregoing methods are consistent with decellularized and irradiated connective tissue matrices. In some embodiments, the tissue implants are also ready to use directly out of the package and do not require rehydration or rinsing.

The tissue implants produced by the foregoing methods are also terminally sterile and essentially virus free. The tissue implants have a Sterility Assurance Level (SAL) of better than 10⁻⁶, which indicates that the present method yields a greater than 6-log reduction in microbial organisms. The tissue implants may exhibit at least 10-log viral reduction.

The present description is further illustrated by the following non-limiting examples.

### Example 1

Porcine dermal tissue was treated using various antiviral treatment formulations to evaluate the effect of the antiviral treatment on porcine dermis and the efficacy of decellularization after the antiviral treatment. Patches of porcine dermis (10 cm×10 cm) were treated with peracetic acid solutions, a hypotonic solution, a decellularization solution, an alkaline alcohol solution, and TRIS rinse, as described in Table 1 below.

TABLE 1

| Process Step | | Arm 1 | Arm 2 | Arm 3 | Arm 4 |
|---|---|---|---|---|---|
| Number of 10 cm × 10 cm | | 5 | | | |
| patches | | | | | |
| Peracetic Acid | Time | 1 | 1 | 1 | N/A |
| (PAA) | (hours) | | | | |
| Treatment | Temp | Room | Room | Room | N/A |
| | | Temp | Temp | Temp | |
| | PAA | 0.05% | 0.05% | 0.05% | N/A |
| | Concentration | | | | |
| | Ethanol | 20% | 20% | 0% | N/A |
| | Concentration | | | | |
| | NaCl | 1M | 0.9% | 0.9% | N/A |
| | Concentration | | | | |

| | |
|---|---|
| Hypotonic | 2-4 hours, 37° C. |
| Decellularization | 1% Triton X-100, 5% Ethanol, |
| | 0.9% Benzyl Alcohol 40 hours, |
| | 37° C. |
| Alkaline Alcohol | 20% Ethanol, 0.02N NaOH, |
| | 1M NaCl 4 hours, 37° C. |
| TRIS Rinse | 2 x, 60-90 minutes, 37° C. |

### Peracetic Acid Formulations

The pH of each of the peracetic acid solutions was 4.8 (Arm 1) and 4.7 (Arms 2 and 3). The formulation of Arm 1 was patterned after the alkaline alcohol solution with a direct substitution of the 0.05% peracetic acid for 0.02N sodium hydroxide. The formulation of Arm 2 was similar to Arm 1, but had a reduced sodium chloride concentration to serve as an isotonic control. The formulation of Arm 3 was similar to Arm 2, but was altered by removal of the ethanol to serve as a solvent control. The patches from Arm 4 were processed without the peracetic acid treatment to serve as a negative control.

### Biomechanical Evaluations

The tensile strength of each of the patches was evaluated after the treatments by taking two dog bone strips (die E1784) in sets perpendicular with respect to each other from each sample. The strips were pulled under uniaxial tension until failure occurred while monitoring an applied load at a fixed elongation rate of 40 mm/min. The average results are summarized in Table 2 below.

TABLE 2

| | | Tensile stress at | Tensile strain | Modulus - |
|---|---|---|---|---|
| | Thickness | max stress | at break | Automatic |
| Arm | (mm) | (MPa) | (mm/mm) | Young's (MPa) |
| 1 | 2.14 ± 0.29 | 16.03 ± 1.84 | 1.95 ± 0.47 | 15.67 ± 3.67 |
| 2 | 2.21 ± 0.23 | 15.04 ± 6.26 | 2.24 ± 0.77 | 14.17 ± 6.62 |
| 3 | 2.35 ± 0.13 | 18.79 ± 5.69 | 2.24 ± 0.54 | 17.00 ± 4.98 |
| 4 | 2.12 ± 0.14 | 22.08 ± 11.16 | 1.80 ± 0.34 | 24.00 ± 13.80 |

There was no notable difference in tensile strength for tissue treated with 0.05% peracetic acid and 1M NaCl (Arm 1) as compared to 0.05% peracetic acid and 0.9% NaCl (Arm 2). There also was no notable difference in tensile strength for tissue treated with 0.05% peracetic acid and 20% ethanol (Arm 2) as compared to 0.05% peracetic acid and no ethanol (Arm 3). The addition of peracetic acid did not appear to cause a change in the tensile strength of the tissue; however, there was much greater variability (increased standard deviation) for both the maximum stress and modulus.

There was no significant difference in tensile strength of the porcine dermis treated with and without the peracetic acid (Arm 1 vs. Arm 4). Tissue treated without peracetic acid provided a higher average value for maximum tensile stress and a higher Young's Modulus (increased rigidity); however, the values were within the standard deviation of the tissues treated with peracetic acid.

### Histology Analysis

Two sections from one patch from each Arm were submitted to pathology for histology preparation. Unstained sections were provided for immunohistochemistry alpha-gal staining The slides were evaluated microscopically.

The hematoxylin and eosin (H&E) stained slides from each study Arm showed significant reduction of hematoxylin staining relative to the unprocessed control. The three peracetic acid treated materials (Arms 1, 2, 3) were comparable to each other and to the tissues without peracetic acid treatment (Arm 4). The slides from all treatment protocols showed some residual hematoxylin staining in vascular bundles, pill muscle, and around hair follicles. Additionally, examination of the matrix from all treatment protocols showed retention of normal collagen structure and crimp.

The results of the immunohistochemistry (IHC) alpha-gal staining were similar to the H&E staining Each treatment protocol showed some reduction of alpha-gal staining relative to the unprocessed control. The three peracetic acid treated materials (Arms 1, 2, 3) were comparable to each other and to the tissues without peracetic acid treatment (Arm 4). The slides from all treatment protocols showed some residual alpha-gal staining in vascular bundles, pill muscle and around hair follicles.

The biomechanical analysis and microscopic evaluation showed no change for treatment with any of the peracetic acid formulations. The histological evaluation showed the peracetic acid treatments did not negatively affect the amount of residual hematoxylin or alpha-gal staining

### Example 2

Porcine dermal tissue was treated using various decellularization formulations to evaluate the effect of the decellularization treatment on porcine dermis. Triton X, a detergent that is commonly used in decellularization processes, was used as a control. Patches of porcine dermis (10 cm×10 cm) were treated with a hypotonic solution, a decellularization solution, an alkaline alcohol solution, and TRIS rinse, as described in Table 3 below.

TABLE 3

| Process Step | | Arm 1 | Arm 2 | Arm 3 | Arm 4 | Arm 5 |
|---|---|---|---|---|---|---|
| Number of 10 cm × | | 8 | | | | |
| 10 cm patches | | | | | | |
| Hypotonic | | 2-4 hours, 37° C. | | | | |
| De- | Time | 40 hours | | | | |
| cellularization | Temp | 37° C. | | | | |
| | EtOH | 5% | 5% | 10% | 5% | 10% |
| | Triton | 1% | 0% | 0% | 0% | 0% |
| | DMSO | 0% | 0% | 0% | 10% | 10% |
| | Benzyl | 0.9% | 0.9% | 0.9% | 0.9% | 0.9% |
| | Alcohol | | | | | |

| | |
|---|---|
| Alkaline Alcohol | 20% Ethanol, 0.02N NaOH, 1M NaCl |
| | 1, 4, 6, and 24 hours, 37° C. |
| TRIS Rinse | 2 x, 60-90 minutes, 37° C. |
| Sodium Ascorbate | overnight, refrigerated |

### Histology Analysis

Patches from each Arm were submitted to pathology for histology preparation. The slides were evaluated microscopically. The hematoxylin and eosin (H&E) stained slides from study Arms 1, 4, and 5 showed significant reduction of hematoxylin staining relative to the unprocessed control while the treatments from Arms 2 and 3 were not effective at reducing hematoxylin staining The results of the immunohistochemistry (IHC) alpha-gal staining were similar to the H&E staining Each Study Arm showed a reduction of alpha-gal staining relative to the unprocessed control. Study Arm 1 showed a greater reduction than Study Arms 4 and 5, which showed a significantly greater reduction than Study Arms 2 and 3.

### Example 3

Porcine dermal tissue was treated using various decellularization formulations to evaluate the effect of the decellularization treatment on porcine dermis. Patches of porcine dermis (10 cm×10 cm) were treated with a hypotonic solution and a decellularization solution, as described in Table 4 below.

TABLE 4

| Process Step | | Arm 1 | Arm 2 | Arm 3 | Arm 4 |
|---|---|---|---|---|---|
| Number of | | 2 patches per arm | | | |
| 10 cm × 10 cm patches | | | | | |
| Hypotonic | | 2-4 hours, 37° C. | | | |
| Decellularization | Time/Temp | 40 hours, | 30 min | 30 min | 16-24 hours, |
| | | 37° C. | | | 37° C. |
| | EtOH | 5% | 5% | 10% | 5% |
| | Triton | 1% | 0% | 0% | 0% |
| | DMSO | 0% | 0% | 0% | 10% |

| | | | | |
|---|---|---|---|---|
| DMSO/saline | 25% | 50% | 50% | N/A |
| | 40 hours, | 30 min | 16-24 hours, | |
| | 37° C. | | 37° C. | |
| DMSO/saline | 50% | 100% | N/A | N/A |
| | 30 min | 16-24 hrs, | | |
| | | 37° C. | | |
| DMSO | 100% | N/A | N/A | N/A |
| | 16-24 hours, | | | |
| | 37° C. | | | |

Higher concentrations of DMSO altered both the tissue appearance and tissue stiffness. The tissue stiffness may be an important characteristic for a specific application of the tissue.

### Example 4

Pilot scale experiments were conducted using two prototype processes to prepare porcine dermal tissue. The processes are summarized in Tables 5 and 6 below.

TABLE 5
DMSO Pilot Scale Treatment Protocol

| | | Actual | Actual |
|---|---|---|---|
| | | Interval | Interval |
| | | Run 1 | Run 2 |
| Process Step | Description | (hr:min) | (hr:min) |
| Amount of Dermis | 5 sheets minimum | n/a | n/a |
| | 20 × 50 cm | | |
| Peracetic Acid Treatment | 0.05% PAA/20% | 01:01 | 01:06 |
| | ETOH/Water, | | |
| | 1 hour room temp. | | |
| Hypotonic | 4-8 hours, 37° C. | 04:09 | 04:07 |
| Decellularization | 24 or 48 hours, 37° C. | 48:01 | 40:35 |
| (5% ETOH/10% DMSO/0.9% | | | |
| Benzyl Alcohol) | | | |
| Alkaline Alcohol | 16-24 hours, 37° C. | 16:03 | 24:14 |
| (0.02N NaOH/20% ETOH/ | | | |
| 1M NaCl) | | | |
| Water Rinse | 2 X Volume | n/a | n/a |
| Laser Cut product | | n/a | n/a |
| Water hold during cutting | | n/a | n/a |
| Peracetic Acid Treatment | 0.05% PAA/Water | 00:31 | 00:34 |
| (Bleaching) | 30 minutes | | |
| TRIS Rinse | 4 minutes | 00:04 | 00:04 |
| TRIS Rinse | 4 minutes | 00:05 | 00:05 |

| | | |
|---|---|---|
| Package | n/a | n/a |
| Terminal Sterilization | n/a | n/a |
| (Gamma Irradiation 25-40 kGy and | | |
| E-beam 25-30 kGy | | |

TABLE 6
Triton Pilot Scale Treatment Protocol

| | | Actual |
|---|---|---|
| | | Interval |
| Process Step | Description | (hr:min) |
| Amount of Dermis | 5 sheets minimum | n/a |
| | 20 × 50 cm | |
| Peracetic Acid Treatment | 0.05% PAA/20% ETOH/Water, | 01:04 |
| | 1 hour room temp. | |
| Hypotonic | 4-8 hours, 37° C. | 04:53 |
| Decellularization | 40 hours, 37° C. | 39:42 |
| (5% ETOH/5% Triton X- | | |
| 100/0.9% Benzyl Alcohol) | | |
| Water Rinse | 2 X Volume | n/a |
| 30% PEG | 1 × 8 hrs, 1 × 16 hrs, | 08:10 |
| | 1 × 8 hrs. 37° C. | 15:53 |
| | | 07:54 |
| Water Rinse | 2 X Volume | n/a |
| Alkaline Alcohol 20% ETOH/ | 16 hours, 37° C. | 16:38 |
| 0.02N NaOH/1M NaCl | | |
| Water Rinse | 2 X Volume | n/a |
| Laser Cut product | | n/a |
| Water hold during cutting | | n/a |
| Peracetic Acid Treatment | 0.05% PAA/Water | 00:34 |
| (Bleaching) | 30 minutes | |
| TRIS Rinse | 4 minutes | 00:04 |
| TRIS Rinse | 4 minutes | 00:04 |

| | |
|---|---|
| Package | n/a |
| Terminal Sterilization | n/a |
| (Gamma Irradiation 25-40 kGy/E-beam 25 kGy) | |

Samples of treated tissue were tested to evaluate the bioburden and cytotoxicity. The results are summarized in Table 7 below.

TABLE 7

| | DMSO Pilot | DMSO Pilot | |
|---|---|---|---|
| | Run 1 | Run 2 | Triton Pilot |
| Bioburden | (Post) 7 cfu/ | (Pre) 10⁶ cfu/mL | (Post) 1 cfu/ |
| (N = 3) | device | | device |
| DNA Conc. | (Pre) 228 ± 62 | (Pre) 98 ± 20 | |
| (n = 6)(ng/mg) | (Post) 24 ± 12 | (Post) 2 ± 1 | (Post) 13 ± 5 |
| LAL (n = 3) | <0.01 EU/mL | <0.01 EU/mL | <0.01 EU/mL |
| Cytotoxicity | Not cytotoxic (0) | n/a | Cytotoxic (4) |
| | | | 1:4 dilution = not |
| | | | cytotoxic (2) |

All pilots produced tissue implants having significantly reduced bioburden and DNA concentrations. The DMSO pilots, however, produced tissue material having no cytotoxicity as compared to the Triton pilot. Thus, use of an aprotic solvent as compared to a detergent provides an improved outcome, passing initial biocompatibility tests and cytotoxicity tests.

The foregoing experiments illustrate that peracetic acid treatment of the tissue materials provided the necessary viral reduction. Moreover, the peracetic acid treatment did not detrimentally impact the tissue viability, as evidenced by the biomechanical evaluation and histological evaluation in Example 1.

It is intended that the foregoing detailed description be regarded as illustrative, rather than limiting, and that it be understood that it is the following claims, including all equivalents, which are intended to define the scope of this invention.

We claim:

1. A method for preparing a biological tissue for implantation comprising:
providing a biological tissue;
contacting the biological tissue with an antiviral treatment formulation comprising an aqueous solution of peracetic acid and an alcohol, wherein the peracetic acid is present in the antiviral treatment formulation at a concentration from about 0.03% to about 1.2% (v/v);
decellularizing the biological tissue; and
decontaminating the biological tissue with an alkaline alcohol solution.

2. The method of claim 1, wherein the peracetic acid is present in the antiviral treatment formulation at a concentration from about 0.03% to about 0.5% (v/v).

3. The method of claim 1, wherein the peracetic acid is present in the antiviral treatment formulation at a concentration from about 0.03% to about 0.1% (v/v).

4. The method of claim 1, wherein the peracetic acid is present in the antiviral treatment formulation at a concentration from about 0.03% to about 0.075% (v/v).

5. The method of claim 1, wherein the alcohol is present in the antiviral treatment formulation at a concentration from about 10% to about 20% (v/v).

6. The method of claim 1, wherein the alcohol comprises ethanol.

7. The method of claim 1, wherein the antiviral treatment formulation comprises sodium chloride at a concentration from about 0.9% to about 1M.

8. The method of claim 1, wherein the biological tissue is treated with the antiviral treatment formulation for a period from about 45 to about 90 minutes.

9. The method of claim 1, wherein the biological tissue is treated with the antiviral treatment formulation at about room temperature.

10. The method of claim 1, wherein the antiviral treatment formulation has a pH from about 4.7 to about 5.0.

11. The method of claim 1, wherein the biological tissue comprises a mammalian tissue.

12. The method of claim 1, wherein the step of decellularizing the biological tissue comprises treating the biological tissue with a decellularization solution comprising ethanol, a polar aprotic solvent, and benzyl alcohol.

13. The method of claim 12, wherein the polar aprotic solvent is selected from the group consisting of acetone, dimethyl sulfoxide, dimethylformamide, dioxane, hexamethylphosphorotriamide, and combinations thereof.

14. The method of claim 12, wherein the polar aprotic solvent is present in the decellularization solution in an amount effective to remove alpha-gal epitopes.

15. The method of claim 12, wherein the polar aprotic solvent comprises dimethyl sulfoxide in the decellularization solution at a concentration from about 10% to about 20% (v/v).

16. A method for preparing a biological tissue for implantation without the use of antibiotics comprising:
providing a biological tissue;
contacting the biological tissue with an antiviral treatment formulation comprising a solution of peracetic acid and an alcohol, wherein the peracetic acid is present in the antiviral treatment formulation at a concentration from about 0.03% to about 1.2% (v/v);
lysing the biological tissue;
decellularizing the biological tissue with a decellularization solution comprising ethanol, dimethyl sulfoxide, and benzyl alcohol; and
decontaminating the biological tissue with an alkaline alcohol solution.

17. The method of claim 16, further comprising bleaching the decontaminated biological tissue with an aqueous solution comprising peracetic acid at a concentration from about 0.03% to about 0.1% (v/v).

18. The method of claim 16, further comprising vacuum packaging the decontaminated biological tissue.

19. The method of claim 18, further comprising terminally sterilizing the vacuum packaged biological tissue.

20. An implantable tissue material prepared according to the method of claim 1.

21. An implantable tissue material prepared according to the method of claim 16.
